(19) United States
(12) Patent Application Publication
(10) Pub. No.: US 2024/0300902 A1
(43) Pub. Date: Sep. 12, 2024

(54) NOVEL TRANSGLUTAMINASE 2 INHIBITOR AND USE THEREOF

(71) Applicant: MD BIOLAB CO., LTD., Seoul (KR)

(72) Inventors: Sang Woo KIM, Seoul (KR); Hyun Cheol CHUNG, Seoul (KR); Eun Yi CHO, Yongin-si (KR); Ye Rin JO, Seoul (KR); Hyeon Joo KIM, Yongin-si (KR); Byeong Hak MOON, Seoul (KR); Yong Chul KIM, Buk-gu (KR); Jin Su BAE, Seongnam-si (KR); Ga Ram KIM, Suncheon-si (KR); Soo Youl KIM, Goyang-si (KR)

(73) Assignee: MD BIOLAB CO., LTD., Seoul (KR)

(21) Appl. No.: 17/913,296

(22) PCT Filed: Nov. 18, 2021
(86) PCT No.: PCT/KR2021/016943

§ 371 Date: Sep. 21, 2022

(51) Int. Cl. C07D 235/10; A61K 31/4184; A61P 35/00

(52) U.S. Cl. CPC C07D 235/10; A61K 31/4184; A61P 35/00

(57) ABSTRACT

The present invention relates to a series of novel benzoimidazole derivatives and their use.

### TECHNICAL FIELD

The present invention relates to a series of novel benzoimidazole derivatives and their use.

### BACKGROUND ART

Cancer is a cell mass composed of undifferentiated cells that ignore the necessary conditions for tissues and undergo unlimited proliferation, as opposed to normal cells that are capable of performing regular and ablative proliferation and inhibition, depending on the individual's needs, and is also referred to as a tumor. Cancer cells performing such unlimited proliferation penetrate into surrounding tissues and, in worst cases, metastasize to other organs of the body, causing severe pain and eventually death of the body. Despite advances in medicine, the number of domestic cancer patients has continued to increase, which results in an increase of about 44% in the last decade, and the global market for anti-cancer drugs has also increased and has been reported to have a volume of about 100 billion dollars a year.

For anticancer therapy, the first-generation chemotherapeutic agents and the second-generation targeted anticancer drugs have been used. Since the development of immunotherapeutic agents as third-generation anticancer drugs to overcome the side effects of the first-generation and second-generation anticancer drugs, research thereon has continued. However, the biggest problem with cancer treatment is cancer recurrence. In addition, with the lack of cancer-specific targets due to the diversity of cancer mutations, there is a problem in that cancer develops to have an anticancer drug resistance during treatment, making cancer treatment difficult. It is also known that the majority of patients die due to metastasis and recurrent cancer even after treatment of the primary cancer. Accordingly, to enhance the efficacy of anticancer agents, a combined use of anticancer agents is suggested.

Transglutaminase (TGase2) is an enzyme that promotes the bonding between the γ-carboxamide group of glutamine residues bound to a specific peptide and various amines. The Tgase2 is known to primarily play a significant role in promoting the prevention, defense, and repair of damage. However, recent studies have reported that abnormal excessive expression may cause the development of various diseases, such as neurodegenerative diseases, atherosclerosis, inflammatory diseases, and autoimmune diseases. In particular, it is reported that the expression of the TGase2 polymerizes and destabilizes p53, resulting in the extinction of p53, and it is reported that TGase2 inhibition can exert anticancer effects on renal cancer with TGase2 overexpressed (Patent Document 1).

### DOCUMENTS OF RELATED ART

### [Patent Document]

(Patent Document 1) Korean Patent No. 10-1643459 (Jul. 21, 2016)

### DISCLOSURE

### Technical Problem

The inventors have made research and efforts to discover novel small molecule compounds with excellent transglutaminase 2 inhibitory activity. As a result, the inventors have confirmed that a series of benzoimidazole derivatives have significantly superior transglutaminase 2 activity compared to streptonigrin, which is known as a conventional transglutaminase 2 inhibitor.

### Technical Solution

A first aspect of the invention provides a compound represented by Formula 1 or a pharmaceutically acceptable salt thereof:

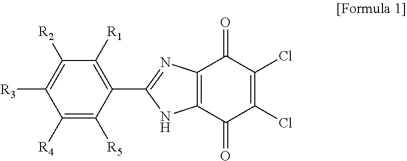

In Formula 1,
R₁ to R₅ are each independently hydrogen, halogen, cyano, nitro, amino, carboxyl, or carbamoyl, except a case where R₁ to R₅ are all hydrogen.

For example, in Formula 1 above, R₁ to R₅ may be each independently hydrogen, or halogen. Specifically, R₁ to R₅ may be each independently hydrogen, bromo, or fluoro. More specifically, the compounds of the invention may include, but are not limited to, a halogen of bromo or fluoro in one or more of R₁ to R₅.

More specifically, the compound may be, but may not be limited to, 2-(4-bromophenyl)-5,6-dichloro-1H-benzo[d]imidazole-4,7-dione or 2-(4-bromo-3-fluorophenyl)-5,6-dichloro-1H-benzo[d]imidazole-4,7-dione).

On the other hand, the compounds of the present invention may exist in the form of pharmaceutically acceptable salts. As the salts, acid addition salts formed by pharmaceutically acceptable free acids may be useful. The term “pharmaceutically acceptable salt” in the present description means any organic or inorganic addition salt of the compound represented by Formula 1, in which the addition salt having a relatively non-toxic and harmless effect on patients, and the side effects attributable to the salt does not detract the beneficial efficacy of the compound represented by Formula 1.

Acid addition salts are prepared by conventional methods. For example, acid addition salts are prepared by dissolving the compound in an excess amount of an acid aqueous solution and precipitating the salt using a water-miscible organic solvent, such as methanol, ethanol, acetone, or acetonitrile. Equal molar amounts of the compound and acid or alcohol (for example, glycol monomethyl ether) in water may be heated, and then the mixture may be evaporated to be dried, or the precipitated salt may be filtered through suction filtration.

In this case, organic acids and inorganic acids can be used as free acids. Examples of the inorganic acid include hydrochloric acid, phosphoric acid, sulfuric acid, nitric acid, tartaric acid, etc. Examples of the organic acid include methanesulfonic acid, p-toluenesulfonic acid, acetic acid, trifluoroacetic acid, maleic acid, succinic acid, oxalic acid, benzoic acid, tartaric acid, fumaric acid, mandaric acid, propionic acid, citric acid, lactic acid, glycollic acid, gluconic acid, galacturonic acid, glutaric acid, glucuronic acid, aspartic acid, carbonic acid, vanillic acid, hydroiodic acid, etc. but are not limited thereto.

In addition, bases can be used to produce pharmaceutically acceptable metal salts. An alkaline metal or alkaline earth metal salt may be prepared, for example, by dissolving the compound in an excessive amount of an alkaline metal hydroxide or alkaline earth metal hydroxide solution, filtering the non-soluble compound salt, and evaporating and drying the filtrate. In this case, as the metal salt, it is pharmaceutically suitable to prepare a sodium, potassium, or calcium salt, but the metal salt is not limited thereto. In addition, a silver salt as the metal salt may be obtained by reacting an alkaline metal or alkaline earth metal salt with a suitable anionic salt (for example, silver nitrate).

Pharmaceutically acceptable salts of the compounds of the present invention include salts of acidic or basic groups that may be present in the compounds of Formulae 1 to 3, unless otherwise indicated. For example, the pharmaceutically acceptable salts of hydroxy groups may include sodium, calcium, and potassium salts, and the pharmaceutically acceptable salts of amino groups include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate). These salts may be prepared by salt preparation methods that are known in the art.

The salts of the compounds of Formula 1 to 3 of the present invention are pharmaceutically acceptable salts, all of which are usable without limitation if they exhibit pharmacological activity equivalent to that of the compounds of Formula 1, that is, if they inhibit aggregation and or hyperphosphorylation of the tau protein.

In addition, the compounds represented by Formula 1, according to the present invention, include, without limitation, not only pharmaceutically acceptable salts thereof, but also all possible solvates such as hydrates that can be prepared therefrom and all possible stereoisomers. The solvates and stereoisomers of the compounds represented by Formula 1 can be prepared from the compounds represented by Formula 1 using methods known in the art.

Further, the compounds represented by Formula 1, according to the present invention, may be prepared in a crystalline form or an amorphous form. When the compounds are prepared in a crystalline form, the compounds may be optionally hydrated or solvated. In the present invention, there may be included not only stoichiometric hydrates of the compounds represented by Formula 1 but also compounds containing various amounts of water. The solvates of the compounds represented by Formula 1, according to the present invention, include both stoichiometric and non-stoichiometric solvates.

A second aspect of the present invention provides a pharmaceutical composition for preventing or treating transglutaminase 2-related diseases, the composition including a compound represented by Formula 1 shown below or a pharmaceutically acceptable salt of the compound.

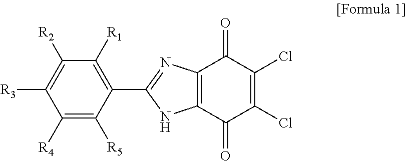

In Formula 1,
R₁ to R₅ are each independently hydrogen, halogen, cyano, nitro, amino, carboxyl, or carbamoyl.

For example, the pharmaceutical composition of the present invention may contain, not limitedly, 5,6-dichloro-2-phenyl-1H-benzo[d]imidazole-4,7-dione, 2-(4-bromophenyl)-5,6-dichloro-1H-benzo[d]imidazole-4,7-dione, or 2-(4-bromo-3-fluorophenyl)-dichloro-1H-benzo[d]imidazole-4,7-dione, as an active ingredient.

The term “prevention” in the present description means any act of inhibiting or delaying the development, spread, or recurrence of diseases induced by abnormal expression and/or aberrant activity of transglutaminase 2 by the administration of the pharmaceutical composition of the present invention. The term “treatment” means any act of improving or beneficially altering the symptoms of diseases by the administration of the pharmaceutical composition of the present invention.

Since the compound of the present invention can efficiently inhibit the activity of transglutaminase 2, the pharmaceutical composition including the compound of the present invention as an active ingredient can be used for the prevention or treatment of diseases that may be caused by overexpression and/or aberrant activity of the transglutaminase 2. The transglutaminase 2-related diseases that can be prevented or treated by the pharmaceutical composition of the present invention include: fibroproliferative diseases such as progressive kidney disease, pulmonary fibrosis, systemic sclerosis, liver cirrhosis, and cardiovascular disease; cancers such as colorectal cancer, breast cancer, pancreatic cancer, ovarian cancer, esophageal squamous cell cancer, glioblastomas, malignant melanomas, renal carcinomas, cervical cell carcinomas, hepatocellular carcinoma, and intrauterine cervical cancer; cardiovascular diseases (CVDs) such as coronary heart disease, deep vein thrombosis, vascular calcification, cerebrovascular diseases, peripheral arterial diseases, rheumatic heart disease, and congenital heart disease; celiac disease; gastroenterological diseases; celiac disease; gastroenterological diseases; inflammatory diseases; neurological disorders such as Alzheimer's disease, Parkinson's disease, supranuclear palsy, Huntington's disease, and polyglutamine diseases; and idiopathic inflammatory myopathies such as dermatitis (DM), polymyositis (PM), and sporadic inclusion body myositis (s-IBM), all of which are caused by abnormal expression and/or aberrant activity of transglutaminase 2. For example, the pharmaceutical composition of the present invention can be used for the prevention or treatment of myocardial infarction or renal cancer. However, the applicable diseases are not limited thereto.

The pharmaceutical composition of the present invention can exert a prophylactic or therapeutic effect by inhibiting the activity of transglutaminase 2. Furthermore, the pharmaceutical composition of the present invention may exhibit anti-cancer effects through p53 restoration obtained by the inhibition of transglutaminase 2.

In a specific example of the present invention, it was found that the pharmaceutical composition containing the compound of the present invention as an active ingredient exhibits a transglutaminase 2 inhibitory activity that is more than twice as good as streptonigrin (KN383), which is a known transglutaminase 2 inhibitor, when used at the same dose (see FIG. 1). In addition, it was also confirmed that the pharmaceutical composition of the present invention caused concentration-dependent restoration of p53 in cancer cell lines (see FIG. 3). Furthermore, it was confirmed that in tumor animal models prepared by infusion of ACHN cells, the tumor volume increase rate was significantly decreased depending on whether the compound of the invention was administered and/or the dose of the compound (see FIG. 4).

For example, the composition of the invention may further contain a pharmaceutically acceptable carrier, diluent, or excipient. The composition of the invention may be formulated by conventional methods and used in a variety of forms, including injectables such as sterile injectable solutions, and oral formulations such as powders, granules, tablets, capsules, suspensions, emulsions, syrups, and aerosols, for each purpose of use thereof. Examples of the carrier, excipient, and diluent suitable for formulation include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil. In addition, a filler, an anti-agglomeration agent, a lubricant, a wetting agent, a flavoring agent, a preservative, and the like may be additionally contained in the composition of the present invention.

Solid formulations for oral administration include tablets, pills, powders, granules, capsules, and the like. These solid formulations are formulated by adding at least one or more excipients such as starch, calcium carbonate, sucrose, lactose, gelatin, and the like to the composition. In addition, in addition to simple excipients, lubricants such as magnesium stearate, talc, or the like may be used.

Examples of the oral liquid preparation may include suspensions, solutions, emulsions, and syrups. In addition to water and liquid paraffin, which are commonly used simple diluents, various excipients such as wetting agents, sweetening agents, fragrances, preservatives, and the like may be included in oral liquid preparations.

Formulations for parenteral administration include aqueous solutions, non-aqueous solutions, suspensions, emulsions, lyophilized formulations, and suppositories. As the non-aqueous solvent and the suspending agent, propylene glycol, polyethylene glycol, vegetable oils such as olive oil, injectable esters such as ethylolate, and the like may be used. As the base of a suppository, Witepsol, Macrogol, Twin61, Kakaoji, Laurinji, Glycerogeratin, and the like may be used. On the other hand, injectables may include conventional additives such as solubilizers, isotonic agents, suspending agents, emulsifiers, stabilizers, preservatives, and the like.

The formulations may be prepared by conventional mixing, granulating, or coating and may contain the active ingredient in a concentration in the range of about 0.1% to 75% by weight, and preferably in the range of about 1% to 50% by weight. The unit dosage form for mammals weighing about 50 to 70 kg contains about 10 to 200 mg of the active ingredient.

In this case, the composition of the present invention is administered in a pharmaceutically effective dosage. In the present invention, the pharmaceutically effective dosage means an amount that is sufficient to treat a disease at a reasonable benefit/risk ratio applicable to medical treatment and not to cause side effects. The effective dosage depends on several factors such as the health condition of the patient, the type and severity of a disease of the patient, drug activity on the disease, a patient's sensitivity to the drug, administration time, administration route, excretion rate, treatment period, combination, and co-used drugs, and also depends on other well-known medical factors. The composition according to the present invention may be administered as an individual therapeutic agent or administered in combination with other therapeutic agents. When administered in combination, the composition and other therapeutic agents may be administered sequentially or simultaneously. The composition may be administered as a single dose or multiple doses. In consideration of all of the above factors, it is important to administer a dosage that can obtain the maximum efficacy with a minimum amount without side effects, and the dosage can be easily determined by those skilled in the art.

For example, the dosage does not in any way limit the scope of the present invention because it can be increased or decreased depending on the route of administration, the severity of the disease, the sex, the weight, the age, etc.

The preferred dosage of the compound of the invention varies depending on the patient's condition and weight, the severity of the disease, the form of the drug, and the route and duration of administration, but may be appropriately selected by those skilled in the art. However, for desirable effects, it is recommended to administer the compound of the invention at a dose of 0.0001 to 100 mg/kg (body weight) per day, and preferably a dose of 0.001 to 100 mg/kg (body weight). Administration may be made via the oral or parenteral route once or multiple times a day.

A third aspect of the present invention provides a composition for inhibiting transglutaminase 2, the composition including a compound represented by Formula 1 shown below or a pharmaceutically acceptable salt of the compound, as an active ingredient.

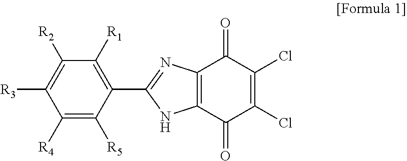

In Formula 1,
R₁ to R₅ are each independently hydrogen, halogen, cyano, nitro, amino, carboxyl, or carbamoyl.

### Advantageous Effects

The benzoimidazole derivatives of the present invention may exhibit markedly enhanced activity compared to streptonigrin, which is a conventional transglutaminase 2 inhibitor, and therefore may be useful for the prevention or treatment of diseases caused by overexpression and/or aberrant activity of transglutaminase 2.

### DESCRIPTION OF DRAWINGS

FIGS. 1a and 1b show the results of a transglutaminase 2 enzyme assay for LDD3732, LDD3922, and LDD3959.

FIG. 2 shows the results of a sulforhodamine B assay for LDD3732, LDD3922, and LDD3959.

FIG. 3 shows the results of a p53 rescue test for LDD3732, LDD3922, and LDD3959.

FIG. 4 shows changes in tumor volume by administration of LDD3959 administration according to administration doses in a preclinical xenograft tumor model.

FIG. 5 shows the results of a transglutaminase 2 enzyme assay for LDD3959, using the colorimetric method.

FIG. 6 shows the results of a cell viability test for LDD3959 in various renal cancer cell lines.

FIG. 7 shows the results of a test of assaying transglutaminase 2 enzyme expression levels in various renal cancer cell lines.

FIG. 8 shows the results of a test of examining the cell proliferation inhibition effect and the angiogenesis inhibition effect of LDD3959 on ACHN, which is a renal cancer cell line.

FIG. 9 shows the results of a test of examining the antitumor effect on mice according to in vivo oral administration of LDD3959 after transplantation of ACHN renal cancer cells, in which reduction in tumor volume is confirmed.

FIGS. 10a to 10g show the antitumor status and hematologic status of mice when the mice were intraperitoneally administered with LDD3959 after CAKI-1 renal cancer cells are transplanted into the mice.

FIG. 11 shows the results of a test of assaying transglutaminase 2 enzyme expression levels in various breast cancer cell lines.

FIGS. 12a to 12c show the results of a cell viability test for LDD3959 according to concentration in various breast cancer cell lines.

FIG. 13 shows the results of a cell viability test for LDD3959 according to time in various breast cancer cell lines.

FIG. 14 shows the results of a protein modification test according to the time-dependent administration of LDD3959 in various breast cancer cell lines.

FIG. 15 shows the results of a test of examining the antitumor effect on mice according to in vivo oral administration and intraperitoneal administration of LDD3959 after transplantation of MDA-MB231 breast cancer cells, in which reduction in tumor volume is confirmed.

FIGS. 16a to 16d show the results of a cell viability test for LDD3959 according to concentration in various cancer cells.

FIGS. 17a and 17b show the results of an inhibitory activity test for LDD3959 against various enzymes associated with cancer.

FIGS. 18a to 18d show the results of a test of examining body weight change, major organ weight, and feed intake two weeks after in-vivo single high concentration administration of LDD3959.

FIGS. 19a and 19b show the results of various blood tests of examining toxicity in the body after two weeks of in vivo single administration of LDD3959 at high concentrations.

FIGS. 20a to 20d show the results of a test of examining body weight change, major organ weight, and feed intake after three weeks of daily in vivo administration of LDD3959 at high concentrations.

FIGS. 21a and 21b show the results of various blood tests of examining toxicity in the body after three weeks of daily in vivo administration of LDD3959 at high concentrations.

FIGS. 22a and 22b show the results of various biochemical tests of examining toxicity in the body after three weeks of daily in vivo administration of LDD3959 at high concentrations.

### BEST MODE

Hereinafter, the present invention will be described in detail with reference to examples. However, these examples are intended to describe the present invention in more detail, and the scope of the present invention is not limited to the examples.

### Example 1: Method of Preparing a Series of Benzoimidazole Derivatives (Compounds 5a to 5c)

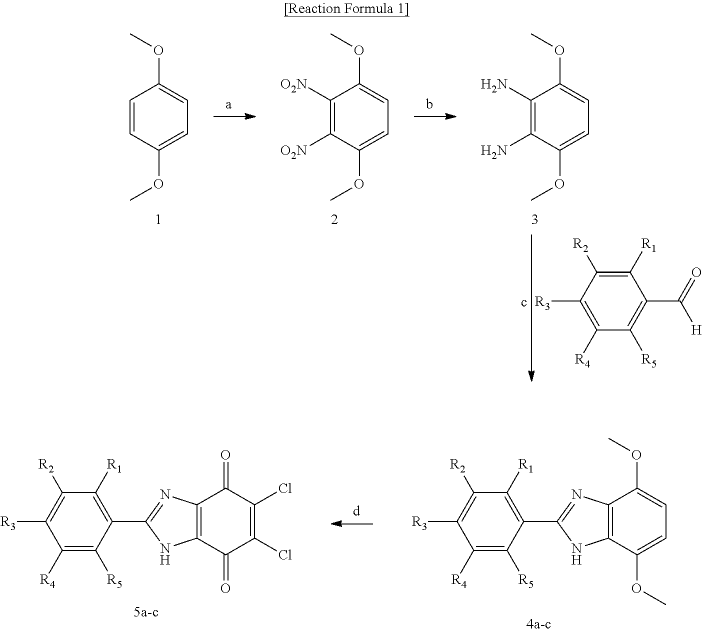

TABLE 1

| No. (LDD code) | R₁ | R₂ | R₃ | R₄ | R₅ |
|---|---|---|---|---|---|
| 4a | H | H | H | H | H |
| 4b | H | H | Br | H | H |
| 4c | H | H | Br | F | H |
| 5a(LDD 3732) | H | H | H | H | H |
| 5b(LDD 3922) | H | H | Br | H | H |
| 5c(LDD 3959) | H | H | Br | F | H |

### Step 1-1: Preparation of 1,4-dimethoxy-2,3-dinitrobenzene (Compound 2)

15 mL of nitric acid was added to 5 g of 1,4-dimethoxybenzene (36.19 mmol), and the mixture was stirred at 0° C. for 1 hour, at room temperature for 1 hour, and then 100° C. for 1 hour. After cooling at room temperature, the mixture was neutralized with 1N NaOH solution at 0° C. and filtered under reduced pressure to obtain a solid product. Purification by silica gel column chromatography (developing solvent: n-hexane to ethyl acetate=4:1) was performed, and the title compound was obtained.

Yield: 79%; 10

¹H NMR (CDCl₃) δ 3.93 (6H, s, CH₃), 7.2 (2H, s, CH);

MS (ESI): m/z=228.68 (M⁺+1).

### Step 1-2: Preparation of 3,6-dimethoxy-1,2-diamine (Compound 3)

The 1,4-dimethoxy-2,3-dinitrobenzene (2 g, 8.76 mmol) prepared according to step 1-1 above was dissolved in 10 mL of methanol, and 100 mg of a Pd/C catalyst was added. The inside of the reactor was substituted with hydrogen gas and stirred at room temperature for 24 hours. The mixture was filtered under reduced pressure, and the obtained filtrate was concentrated under reduced pressure and purified by silica gel column chromatography (developing solvent: n-hexane to ethyl acetate=4:1). Thus, the title compound was obtained.

Yield: 85%;

¹H NMR (CDCl₃) δ 3.51 (4H, s, NH₂), 3.81 (6H, s, CH₃), 6.31 (2H, s, CH);

MS (ESI): m/z=168.73 (M⁺+1)

### Step 1-3: Preparation of 4,7-dimethoxy-2-(unsubstituted or substituted phenyl)-1H-benzo[d]imidazole derivatives (Compounds 4a to 4c)

The 3,6-dimethoxybenzene-1,2-diamine (1 g, 5.94 mmol) prepared according to step 1-2 above was dissolved in 15 mL of toluene, and a series of unsubstituted or substituted benzaldehyde compounds, specifically benzaldehyde, 4-bromobenzaldehyde, 4-bromo-3-fluorobenzaldehyde, (11.88 mmol each) were added for reflux reaction for 6 hours. After cooling at room temperature, extraction with ethyl acetate was performed. The organic layer was dried over anhydrous sodium sulfate and concentrated under reduced pressure. Ethylether was added and then filtered under reduced pressure to obtain the title compounds 4a to 4c) that are in a solid form.

### 4a: 4,7-dimethoxy-2-phenyl-1H-benzo[d]imidazole

Yield: 30%;

¹H NMR (CDCl₃) δ 3.98 (6H, s, CH₃), 6.59 (2H, s, CH), 7.47 (3H, m, phenyl), 8.1 (2H, dd, J=8.1, 1.5 Hz, phenyl);

MS (ESI): m/z=255.07 (M⁺+1)

### 4b: 2-(4-bromophenyl)-4,7-dimethoxy-2-phenyl-1H-benzo[d]imidazole

Yield: 39%;

¹H NMR (CDCl₃) δ 3.97 (6H, s, CH₃), 6.6 (2H, s, CH), 7.61 (2H, d, J=8.7 Hz, phenyl), 7.98 (2H, d, J=8.7 Hz, phenyl);

MS (ESI): m/z=334.8 (M⁺+1).

### 4c: 2-(4-bromo-3-fluorophenyl)-4,7-dimethoxy-2-phenyl-1H-benzo[d]imidazole

Yield: 32%;

¹H NMR (METHANOL-d4) δ 3.96 (6H, s, CH₃), 6.67 (2H, s, CH), 7.75 (1H, m, phenyl), 7.87 (1H, m, phenyl), 8.0 (1H, dd, J=10.1, 2.3 Hz, phenyl);

MS (ESI): m/z=352.79 (M⁺+1).

### Step 1-4: Preparation of 5,6-dichloro-2-(unsubstituted or substituted phenyl)-1H-benzo[d]imidazole-4,7-dione derivatives (Compounds 5a to 5c)

9 mL of Hydrochloric acid and 3 mL of nitric acid were slowly added to 2.0 mmol of the series of unsubstituted or substituted benzaldehyde derivatives prepared according to step 1-2 above, and the mixture was stirred at 80° C. for 1 hour and then at room temperature for 1 hour. The mixture was neutralized with sodium hydrogen carbonate and extracted with ethyl acetate. The organic layer was dried over anhydrous sodium sulfate and concentrated under reduced pressure. Ethylether was added, and then a reduced pressure filtration was performed to obtain the title compounds in a solid form.

### 5a: 5,6-dichloro-2-phenyl-1H-benzo[d]imidazole-4.7-dione

Yield: 33%;

¹H NMR (DMSO-d6) δ 7.55 (3H, m, phenyl), 8.17 (2H, m, phenyl);

MS (ESI): m/z=292.88 (M⁺+1).

### 5b: 2-(4-bromophenyl)-5,6-dichloro-1H-benzo[d]imidazole-4,7-dione

Yield: 40%;

¹H NMR (DMSO-d6) δ 7.77 (2H, m(para), J=8.7, phenyl), 8.11 (2H, m(para), J=8.9, phenyl);

MS (ESI): m/z=372.57 (M⁺+1).

### 5c: 2-(4-bromo-3-fluorophenyl)-5,6-dichloro-1H-benzo[d]imidazole-4,7-dione

Yield: 44%;

¹H NMR (DMSO-d6) δ 7.92 (1H, m, phenyl), 7.96 (1H, m, phenyl), 8.1 (1H, dd, J=10, 1.9 Hz, phenyl);

MS (ESI): m/z=390.42 (M⁺+1).

### Experimental Example 1: In Vitro Transglutaminase 2 Assay (1)

Incorporation of [1,4-14C] putrescine into succinylated casein was measured to determine the inhibitory effect of each compound on the activity of transglutaminase 2 (TGase 2). 1 mU of guinea pig liver-originated transglutaminase 2 (Sigma, St. Louis, MO, USA) was preincubated with various concentrations of GK13 or GK921 in a 0.1 mL reaction buffer with or without 10 mM CaCl₂ for 10 minutes, and 0.4 mL of a substrate solution containing 2% (w/v) succinylated casein and 100 nCi of [1,4-14C] putrescine. After incubation at 37° C. for 1 hour, 4 mL of cold (4° C.) 7.5% (w/v) trichloroacetic acid (TCA) was added to terminate the reaction. The TCA-insoluble precipitate was recovered with a GF/A-grade glass filter (Millipore, Billerica, MA, USA), washed with cold 5% (w/v) TCA, dried, and assessed with a scintillation counter (Beckman Coulter, Brea, CA, USA) to evaluate radiolabel binding. Transglutaminase 2 which was pre-incubated only with a buffer was used as a positive control. The scintillation counts were compared between the example and the positive control, and IC₅₀ values were determined through the logistic linear regression method. The presented data were the mean values of three independent experiments. In this experimental example and the following experimental example, Streptonigrin (described as KN383 in each figure of the drawing), which is an existing transglutaminase 2 inhibitor, was used as a positive control.

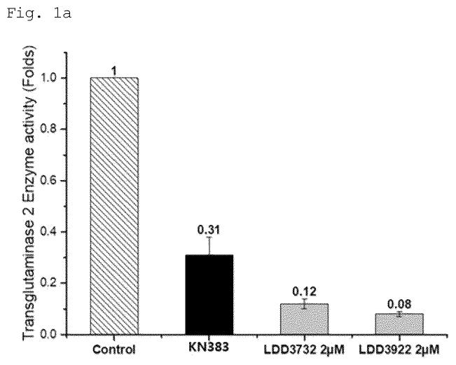

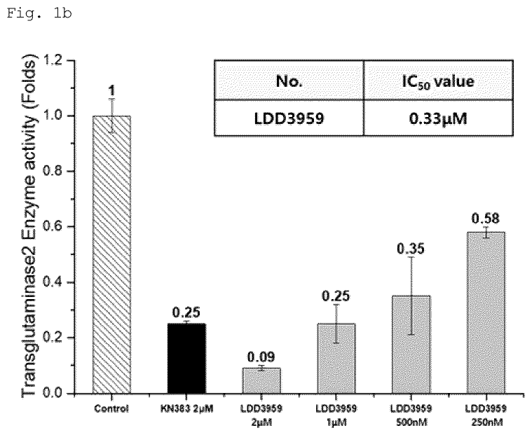

As shown in FIGS. 1a and 1b, for the LDD3732 and LDD3922, the mean value of the negative control was 129.0, the mean value of the control 4147.5, and the mean value of 2 μM streptonigrin was 1275.5. In addition, the mean value of the 2 μM LDD3732 and the mean value of the 2 μM LDD3922 relative to the mean value of the control were determined to be 477.5 and 352.5, respectively, and the fold values were determined to be 0.12 and 0.08, respectively, with standard deviations of 0.02 and 0.01, respectively. For the LDD3959, the mean value of the negative control was 248.5, the mean value of the control was 3606.5, the mean value of the 2 μM LDD3959 was 0.09, and the standard deviation was 0.01. These results indicate that all of the LDD3732, LDD3922, and LDD3959 exhibited two-fold or greater stronger TGase2 enzyme activity inhibitory effects than the control.

### Experiment Example 2: Sulforhodamine B (SRB) Assay

Cells (10,000 cells/well, 100 μL) were incubated in a 96-well microtiter plate. After 24 hours of the incubation, drug (100 μL) was added to each of the wells, and the culture was further incubated at 37° C. for 48 hours. The cells were then fixed in TCA (50 μL per well). The plates were incubated at 4° C. for a minimum of 1 hour or a maximum of 3 hours. The liquid was removed from the plates, and then the plate was washed five times with water and then dried at room temperature (RT) for about 12-24 hours. The fixed cells were stained with 100 μL SRB for 5 minutes at room temperature. After the staining, the plate was washed three times with 1% (w/v) glacial acetic acid and dried at room temperature for about 12-24 hours. Next, the SRB was then dissolved in 10 mM Trizma base, and the absorbance was measured at 515 nm. The effect of the drug was expressed as GI₅₀ (50% growth inhibition).

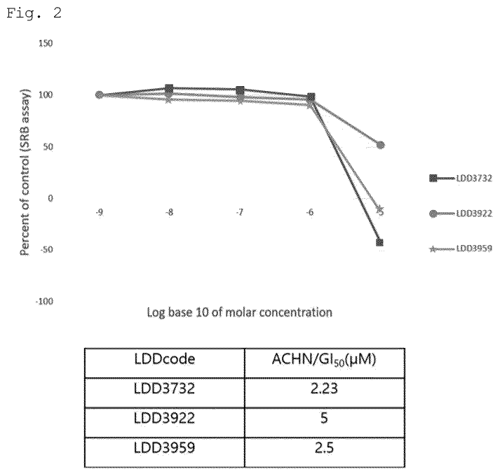

Specifically, each of the LDD3732, LDD3922, and LDD3959 in concentrations of 10⁻⁹, 10⁻⁸, 10⁻⁷, 10⁻⁶, and 10⁻⁵ M were tested, and the percentage decrease in the control value of SRB was measured using an ACHN cancer cell population. As shown in FIG. 2, all three compounds exhibited no significant decrease in cell counts when the concentrations were 10⁻⁶ M or lower, but the measurements were dramatically reduced to −41.43, 52.25, and −11.87 at a concentration of 10⁻⁵ M, respectively. These values were calculated as GI₅₀ values of 2.2 μM, 5.0 μM, and 2.5 μM, respectively. This result indicates that the compounds have a significantly superior level of anti-cancer effect.

### Experiment Example 3: P53 Rescue Test Using Western Blotting

Whole cell lysates were prepared using a radioimmunoprecipitation assay (RIPA) buffer (150 mM sodium chloride, 1.0% (w/v) IGEPAL® CA-630 (NP-40), 0.5% (w/v) sodium deoxycholate, 0.1% (w/v) sodium dodecyl sulfate (SDS), a protease inhibitor cocktail, and 50 mM Tris-HCl (pH 8.0) containing a phosphatase inhibitor cocktail. Protein assays were performed through Bradford protein assay (Thermo Scientific, Waltham, MA, USA) to normalize protein expression. The proteins were resolved by SDS-PAGE and transferred to polyvinylidene difluoride (PVDF) membranes (Merck Millipore, Burlington, MA, USA). The membranes were blocked with 5% (w/v) BSA for 1 hour at room temperature and incubated overnight at 4° C. with labeled antibodies. Membranes were washed in TBS-T for 1 hour at room temperature and incubated together with a horseradish peroxidase (HRP)-bound secondary antibody for 1 hour at room temperature. Finally, the membranes were washed in TBS-T for 1 hour at room temperature and developed with enhanced chemiluminescence.

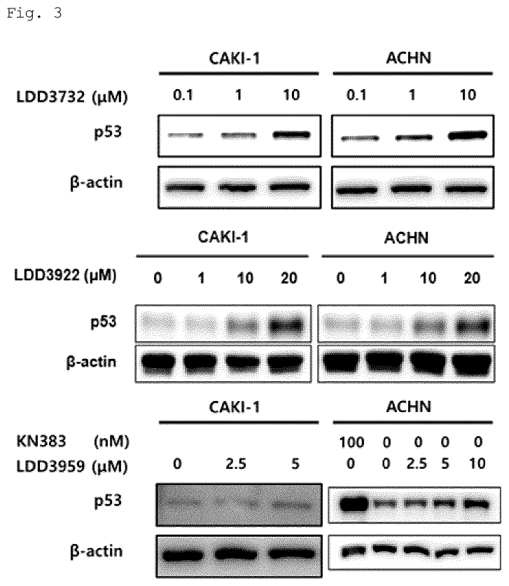

As shown in FIG. 3, all of the LDD3732, LDD3922, and LDD3959 exhibited dose-dependent p53 rescue effects. It was confirmed that the Western blot band of p53 was thickened as the treatment progressed from a relatively low concentration to a relatively high concentration. This indicates that the compounds exert an anti-cancer effect via the route of p53 by inhibiting the TGase2 enzyme activity.

### Experimental Example 4: Preclinical Xenograft Tumor Model

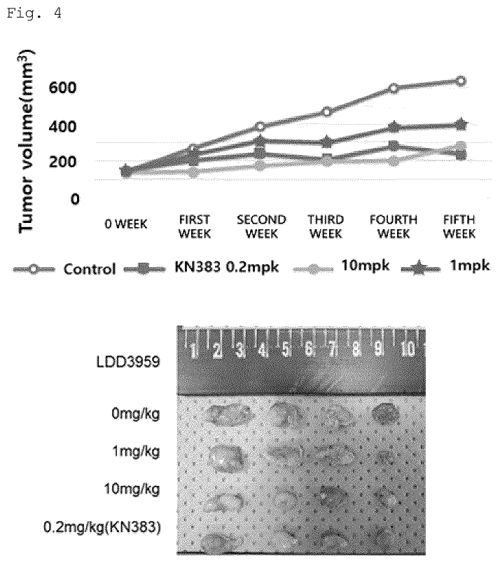

Six-week-old male-specific, pathogen-free BALB/c nude mice (n=20) were purchased from the central lab (Animal Inc., Seoul, Korea). Each mouse was injected subcutaneously with ACHN cells (5.0×10⁶ cells/each). When tumors reached an appropriate size (100 to 150 mm³ for ACHN), mice were randomly selected and classified into 4 groups (n=4 to 5) according to the volume and weight of the tumor: The control group was untreated; the streptonigrin-treated group was administered in an amount of 0.2 mg/kg; and the LDD3959-treated group was administered in an amount of 1 mg/kg or 10 mg/kg. The streptonigrin and LDD3959 were orally administered to each treatment group, once daily for 5 days per week. For tumor identification, the mice were euthanized with 7.5% CO₂ and the tumors were recovered for immunohistochemical analysis. The primary tumor size was measured once per week with calipers and the results are shown in FIG. 4. The tumor volume was calculated according to the following equation: V=(A×B×B)/2 where V is the volume (mm³), A is the long diameter (mm), and B is the short diameter (mm). The experiment was reviewed and approved by the Institutional Animal Care and Use Committee (IACUC) of the National Cancer Center Research Institute (NCCRI). The NCCRI is an AAALAC-accredited facility (AAALAC stands for Association for Assessment and Accreditation of Laboratory Animal Care International) and follows the guidelines (IRB number: NCC-20-520) of the Institute of Laboratory Animal Resources (ILAR). The experiment procedure is shown in Table 2.

TABLE 2

| | |
|---|---|
| Cell line | ACHN/ATCC (Human) |
| Mice | BaLB/c-nude (Orient) |
| N (Head) | 5 |
| Drug delivery | PO/once, daily |
| Treat on/off | On (5) Off (2) |
| Week | 6-7 weeks (expected) |
| | End Point: Control volume - |
| | 1000 mm³ |
| Drug dose | Control |
| | LDD3959 1 mg/kg |
| | LDD3959 10 mg/kg |
| | KN383 0.2 mg/kg |
| Drug vehicle | LDD3959 = 0.5% CMC + 0.1% |
| | tween80 + 99.4% DW |
| | KN383 = 1% DMSO + 99% PBS |

As shown in FIG. 4, the tumor volume increase rate decreased as the LDD3959 dose increased, and the tumor size of the untreated control continued to increase proportionally over time, but the tumor size of the increase rate of the LDD 3959-treated group decreased.

### Experimental Example 5: In Vitro Transglutaminase 2 Assay (2)

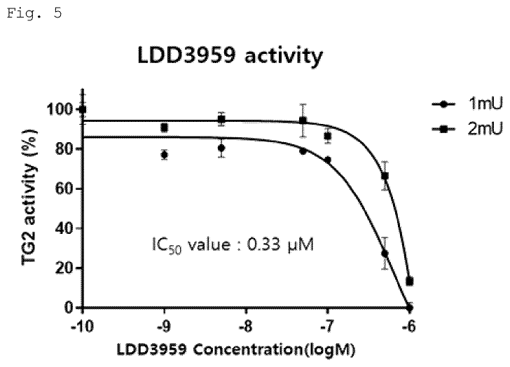

After 10-minute reactions of LDD3959 and 1 mU and 2 mU of transglutaminase 2 originating in guinea pig liver at 37° C. at each concentration, 80 μL of a substrate mixture (500 nM γ-glutamyl donor substrate, 400 mM MES (4-Morpholineethanesulfonic acid), 200 mM of DL-Dithiothreitol (DTT), and 200 mM of calcium chloride (CaCl₂) were added thereto. After 2-hour reactions at 37° C., 0.4 ml of a stop solution (0.37 M of iron (III) chloride hexahydrate (FeCl₃), 0.67 M of hydrochloric acid (HCl), and 0.2 M trichloroacetic acid (TCA) were added thereto to terminate the reaction. After recovering the precipitate with a microcentrifuge, the precipitate was dispensed to the 96-well microplate by 200 μL, and the absorbance was measured at 525 nm. The results are shown in FIG. 5.

Referring to FIG. 5, it is possible to confirm that the LDD3959 exhibits a similar level of transglutaminase 2 inhibitory to that of Experimental Example 1.

### Experimental Example 6: Examination of Cell Viability in Various Renal Cancer Cell Lines

Cells (20,000 cells/well, 100 μl) were incubated in a 96-well microplate. After 24 hours of the incubation, LDD3959 cells were added to each well in various concentrations by using a serum-free cell culture medium, and the cells were cultured at 37° C. in the presence of 5% CO₂ for 2 hours. Next, the existing culture medium was replaced with a culture medium containing 10% FBS, and then incubation was carried out again for 48 hours. The existing culture medium was removed, and a new culture medium containing a water soluble tetrazolium salt was added. After that, the plate was incubated at 37° C. in the presence of 5% CO₂ for a duration in the range of 1 hour to 3 hours, and then absorbance was measured at 450 nm.

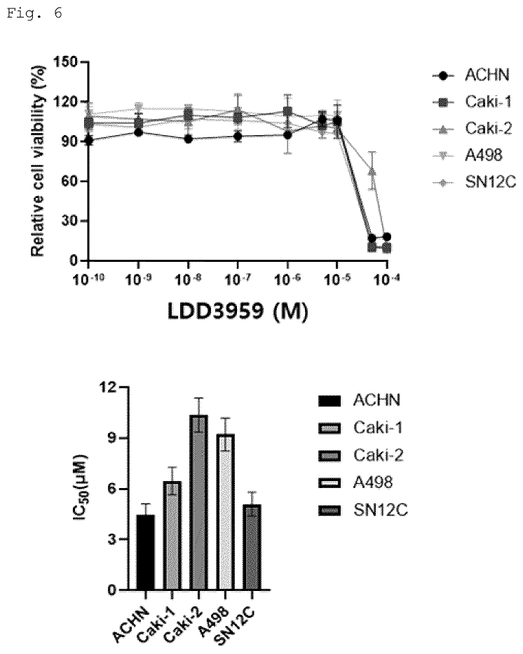

As a result, it was confirmed that cancer cells in a variety of renal cancer cells were killed, as shown in FIG. 6.

### Experiment Example 7: Analysis of Transglutaminase 2 Protein Expression in Several Renal Cancer Cell Lines

A solution obtained by adding a protease inhibitor cocktail and a phosphatase inhibitor cocktail to RIPA buffer (25 mM Tris·HCl pH 7.6, 150 mM NaCl, 1% NP-40, 1% sodium deoxycholate, and 0.1% SDS) was used to prepare whole cell lysates of multiple renal cancer cells. Next, the protein was quantified using a bicinchoninic acid (BCA) protein assay, and the protein was resolved by SDS-PAGE and transcribed into a polyvinylidene difluoride (PVDF) membrane. The membrane was then blocked with 5% BSA for 1 hour at room temperature, reacted with the primary antibody of anti-transglutaminase 2 overnight at 4° C., washed in PBS-T for 30 minutes at room temperature, and reacted with horseradish peroxidase (HRP)-bound secondary antibody for 2 hours at room temperature. Finally, the membranes were washed in PBS-T for 30 minutes at room temperature and developed with enhanced chemiluminescence to determine the expression level of the protein.

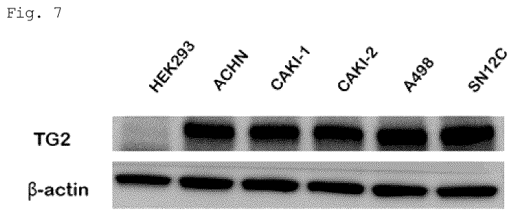

Referring to FIG. 7, the results show that transglutaminase 2 was not overexpressed in HEK293, which are normal cells, but was overexpressed in all renal cancer cells when transglutaminase 2 expression was identified for several types of renal cancer cells. In other words, to kill cancer cells, it is necessary to suppress the transglutaminase 2 expression.

### Experiment Example 8: Examination of Expression of Cell Proliferation Inhibition-Related Proteins and Angiogenesis Inhibition-Related Proteins in Renal Cancer Lines, Using LDD3959

LDD3959 treatment at various concentrations was performed on ACHN cells, using a serum-free culture medium, and a reaction was performed at 37° C. in the presence of 5% CO₂ for 2 hours. The culture medium was replaced with a culture medium containing 10% FBS, and a reaction was performed for 48 hours. The culture medium was removed, and PBS washing was performed. With the use of a solution obtained by adding a protease inhibitor cocktail and a phosphatase inhibitor cocktail to RIPA buffer (25 mM Tris*HCl pH 7.6, 150 mM NaCl, 1% NP-40, 1% sodium deoxycholate, and 0.1% SDS) was used to prepare whole cell lysates.

The proteins were quantified by Bicinchoninic acid (BCA) protein assay. The proteins were resolved by SDS-PAGE and transcribed into a polyvinylidene difluoride (PVDF) membrane. The membrane was then blocked with 5% BSA for 1 hour at room temperature, allowed to react with a labeled antibody overnight at 4° C., washed in PBS-T for 30 minutes at room temperature, and allowed to react with a horseradish peroxidase (HRP)-bound secondary antibody for 2 hours at room temperature. Finally, the membrane was washed in PBS-T for 30 hours at room temperature and developed with enhanced chemiluminescence.

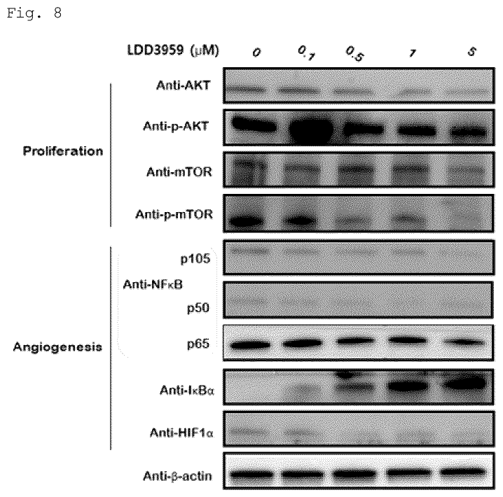

Referring to FIG. 8, the assay results show that two main mechanisms are identified when cell death is caused by the inhibition of transglutaminase 2. The first mechanism is inhibiting cell proliferation by inhibiting Akt/mTOR45 signals, and the second mechanism is inhibiting angiogenesis and transcription to cellular environmental factors by inhibiting NFκB complexes.

### Experiment Example 9: Validation of Antitumor Effects in In Vivo Renal Cancerous Tumors

To examine the antitumor effect of LDD3959, CanN.Cg-Foxn1nu/CrljOri mice were used as experimental animals. The mice were xenografted with ACHN renal cancer cells or CAKI-1 renal cancer cells to induce tumors. The ACHN renal cancer cell-transplanted group was orally administered with LDD3959, and the CAKI-1-transplanted group was intraperitoneally administered with LDD3959. In this test, the oral administration was performed daily and the intraperitoneal administration was performed twice a week.

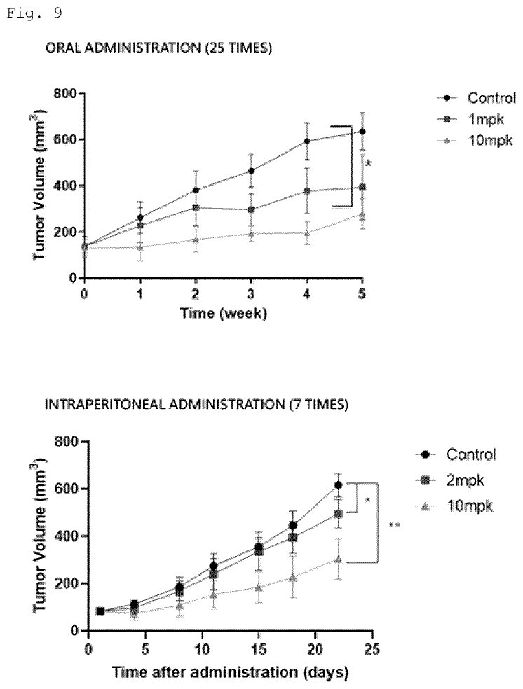

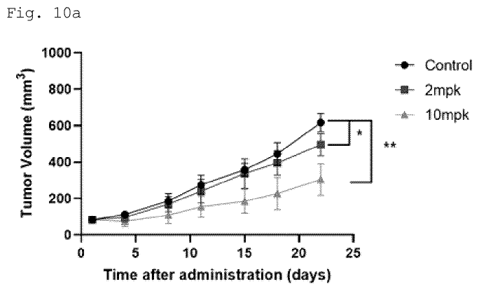

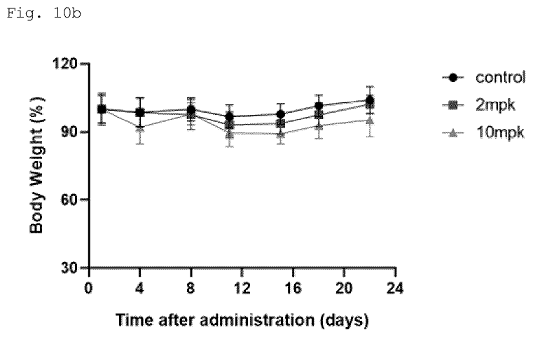

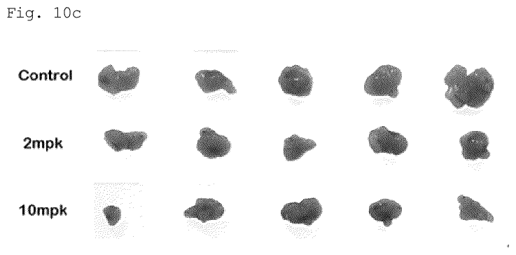

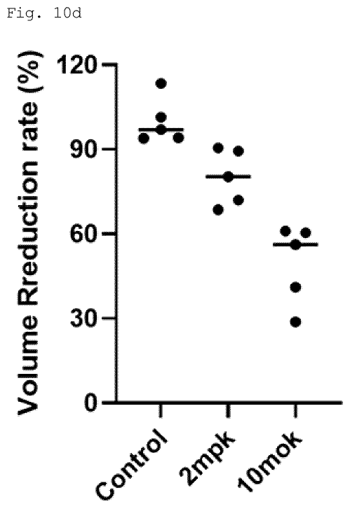

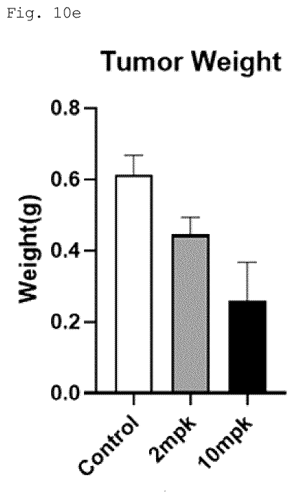

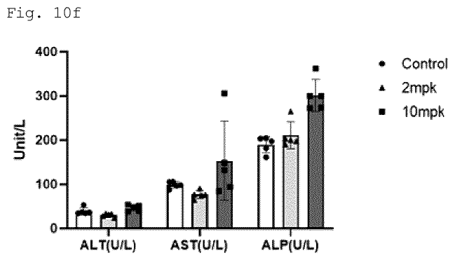

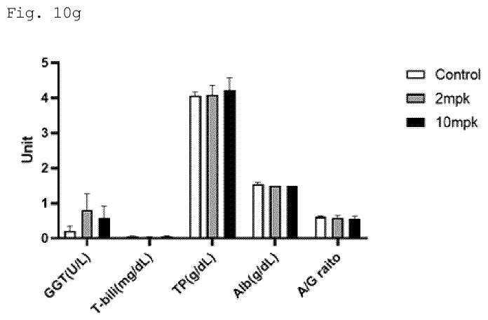

Referring to FIGS. 9 and 10, the results show that both of the oral administration and the intraperitoneal administration exhibited concentration-dependent tumor growth inhibitory effects in the LDD3959-administered group, and no abnormal findings were identified by the hematological examination in the intraperitoneal administration group.

### Experiment Example 10: Comparison in Transglutaminase 2 Protein Expression Level Among Several Renal Cancer Cell Lines

A solution obtained by adding a protease inhibitor cocktail and a phosphatase inhibitor cocktail to RIPA buffer (25 mM Tris·HCl pH 7.6, 150 mM NaCl, 1% NP-40, 1% sodium deoxycholate, and 0.1% SDS) was used to prepare whole cell lysates of several types of breast cancer cells.

For examination of transglutaminase 2 protein expression, proteins in the prepared whole cell lysates were quantified using a bicinchoninic acid (BCA) protein assay, the proteins were resolved by SDS-PAGE and transcribed into polyvinylidene difluoride (PVDF) membranes, the membranes were blocked with 3% BSA for 1 hour at room temperature, and the primary antibody of anti-transglutaminase 2 was allowed to react overnight at 4° C. Next, the membranes were washed in PBS-T for 1 hour at room temperature and incubated together with a horseradish peroxidase (HRP)-bound secondary antibody for 1 hour at room temperature.

Finally, the membranes were washed in PBS-T for 30 minutes at room temperature and developed with enhanced chemiluminescence to determine the expression level of the protein.

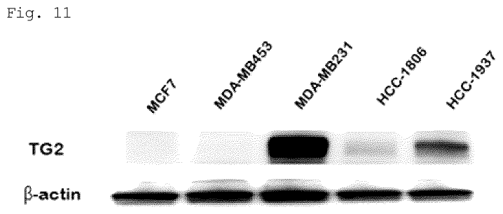

Referring to FIG. 11, the results confirmed that the expression level of the transglutaminase 2 was elevated in the breast cancer cells. Particularly, in triple negative breast carcinoma cells in which none of the estrogen receptor (ER), progesterone receptor (PRs), and human epidermal growth factor receptor 2 (Her2) protein were expressed, the expression level of the transglutaminase 2 was high.

### Experimental Example 11: Comparison in Cell Viability Among Breast Cancer Cell Lines According to LDD-3959 Treatment

First breast cancer cells (MDA-MB-453, HCC-1806, HCC-1937: 10,000 cells/well, 100 μl) and second breast cancer cells (MDA-MB-231, MCF-7: 10,000 cells/well, 100 μl) were cultured in 96-well microplates.

After 24 hours, LDD3959 cells of various concentrations were added to each well, using a serum-free cell culture medium, and a reaction was allowed to proceed for 2 hours at 37° C. in the presence of 5% CO₂. The culture medium was replaced with a culture medium containing 10% FSB, and a reaction was performed for 72 hours.

The existing culture medium was removed, and a new culture medium containing a water soluble tetrazolium salt was added. After that, the plate underwent a reaction at 37° C. in the presence of 5% CO₂ for a duration in the range of 1 hour to 3 hours, and then absorbance was measured at 450 nm.

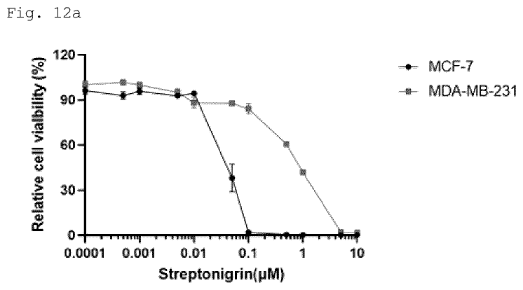

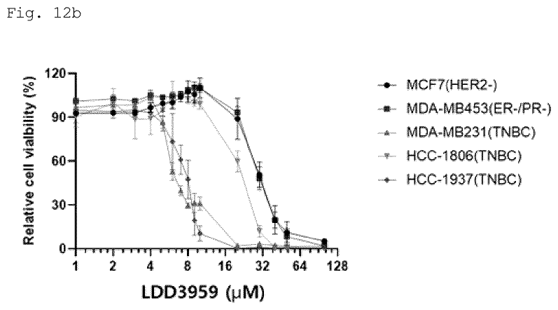

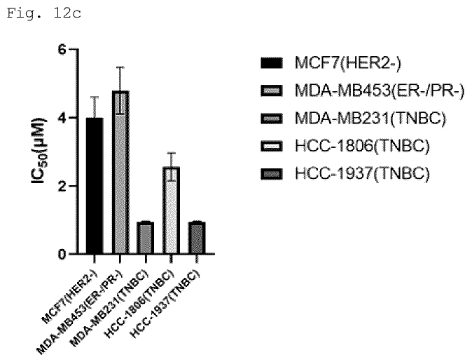

From the results shown in FIGS. 12a to 12c, it was confirmed that when using streptonigrin, which is known as a transglutaminase 2 inhibitor, the effective concentration for apoptosis in MCF7 cells exhibiting no expression of the transglutaminase 2 was low. In the case of using LDD3959, the higher the expression level of the transglutaminase 2, the higher the effective concentration for apoptosis. In particular, in the MDA-MB231 cells exhibiting the highest expression level of the transglutaminase 2, the effective concentration was lowest, and the efficacy was best.

These results indicate that LDD3959 can act at lower concentrations than conventional transglutaminase 2 inhibitors and thus LDD3959 can be used as an advanced anticancer agent with fewer side effects.

### Experimental Example 12: Comparison in Cell Viability Among Breast Cancer Cell Lines According to LDD-3959 Treatment Time

Breast cancer cells (50,000 cells/well, 100 μl) were cultured in 96-well microplates, and after 24 hours, LDD3959 was added to each well in various concentrations, using a serum-free cell culture medium. In each well, a reaction was performed at 37° C. in the presence of 5% CO₂ for 2 hours.

Next, the existing culture medium was replaced with a culture medium containing 10% FSB, and 24, 48, and 72 hours of reactions were performed. After the completion of each reaction, the exiting culture medium was removed, a culture medium containing a water soluble tetrazolium (WST) salt was added, the plate was allowed to react at 37° C. in the presence of 5% CO₂ for a duration in the range of 1 hour to 3 hours, and the absorbance was measured at 450 nm.

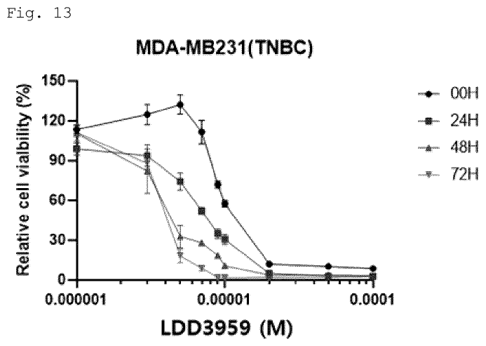

As a result, as shown in FIG. 13, since the MDA-MB231 breast cancer cells are low drug reactive cells, the MDA-MB231 cells exhibited a low apoptosis level at the initial stage, but the apoptosis level increased as the transglutaminase 2 was inhibited, and the signal was transferred.

### Experimental Example 13: Comparison in Protein Expression Level Change Among TNBC Breast Cancer Cell Lines According to LDD-3959 Treatment Time

LDD3959 treatment was performed at various concentrations on the MDA-MB-231 cells, using a serum-free culture medium, and a reaction was performed at 37° C. in the presence of 5% CO₂ for 2 hours. The culture medium was replaced with a culture medium containing 10% FBS, and 24, 48, and 72 hours of reactions were performed. The culture medium was removed, and PBS washing was performed. With the use of a solution obtained by adding a protease inhibitor cocktail and a phosphatase inhibitor cocktail to RIPA buffer (25 mM Tris·HCl, pH 7.6, 150 mM NaCl, 1% NP-40, 1% sodium deoxycholate, and 0.1% SDS) was used to prepare whole cell lysates of diverse types of breast cancer cells.

Next, the proteins were quantified by a bicinchoninic acid (BCA) protein assay. The proteins were resolved by SDS-PAGE and transcribed into polyvinylidene difluoride (PVDF) membranes. The membranes were blocked with 3% (w/v) BSA for 1 hour at room temperature and allowed to react with the primary antibody of anti-transglutaminase 2 overnight at 4° C. The membranes were washed in PBS-T for 30 minutes at room temperature and allowed to react with a horseradish peroxidase (HRP)-bound secondary antibody for 1 hour at room temperature. Finally, the membranes were washed in PBS-T for 30 hours at room temperature and developed with enhanced chemiluminescence.

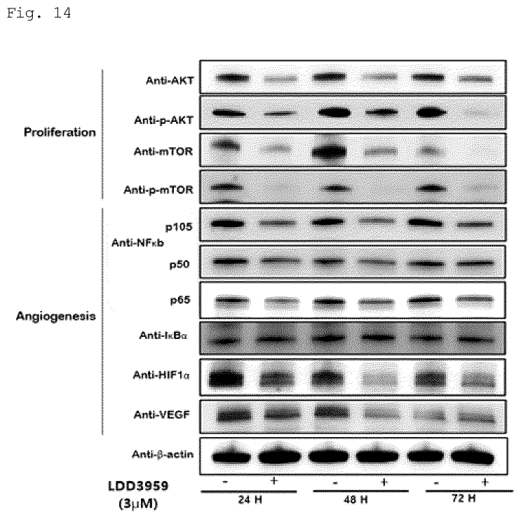

Referring to FIG. 14, from the assay results, it was confirmed that when the transglutaminase 2 was inhibited to cause apoptosis, AKT/mTOR signals were inhibited to cause inhibition of cell proliferation, and NFκB complexes were inhibited to cause inhibition of angiogenesis and inhibition of transcription to cellular environmental factors.

### Experiment Example 14: Examination of Antitumor Effects in In Vivo Breast Cancerous Tumors

To examine the antitumor effect of LDD3959, CanN.Cg-Foxn1nu/CrljOri mice were used as experimental animals. The mice were xenografted with MDA-MB231 breast cancer cells to induce tumors. The MDA-MB231 breast cancer cell-transplanted group was divided into two groups: an oral administration group and an intraperitoneal administration group. In this test, the oral administration was performed daily and the intraperitoneal administration was performed twice a week.

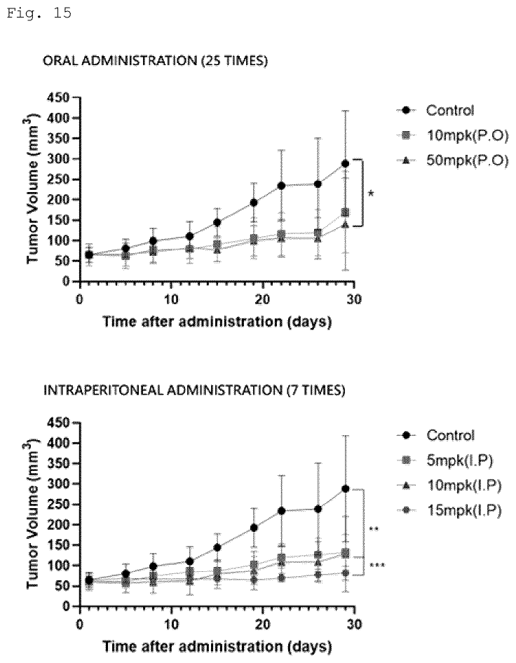

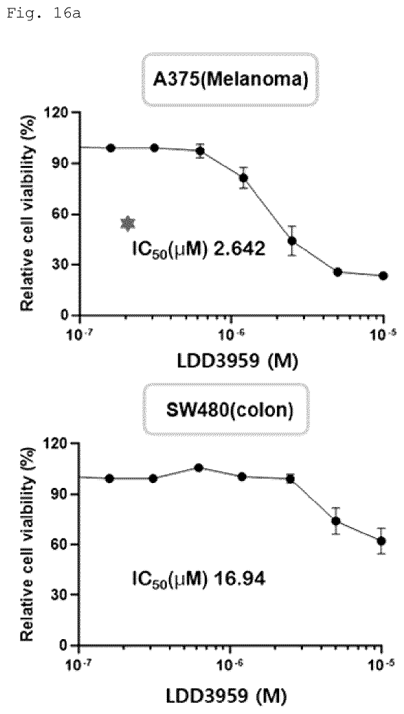

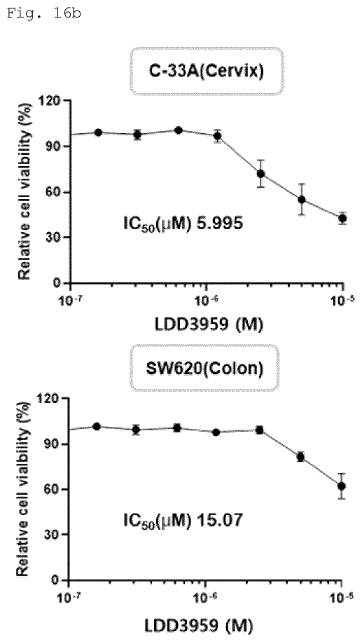

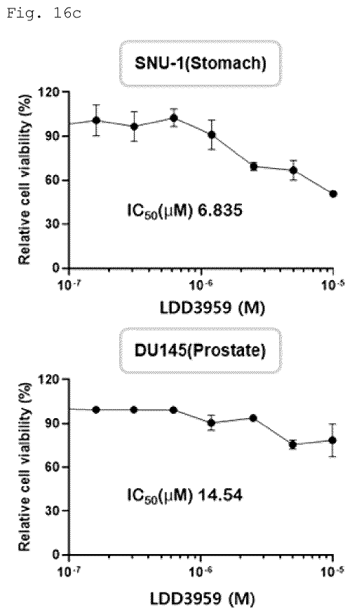

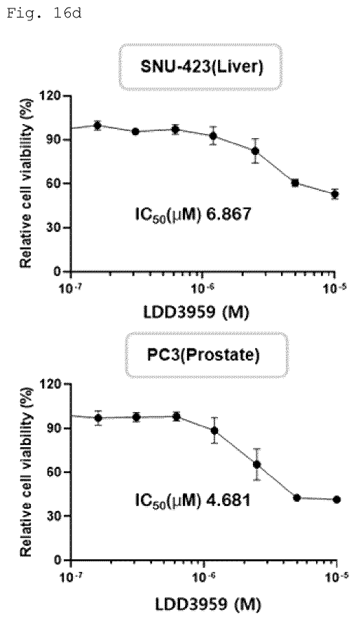

As a result, it was confirmed that both of the administration method exhibited concentration-dependent antitumor effects, as shown in FIG. 15.

### Experimental Example 15: Examination of Antitumor Action in Various Tumors

The OncoPanel™ assay conducted by Eurofins confirmed the antitumor efficacy of LDD3959 in a total of 8 tumors at concentrations of 10 μM or lower.

### Experimental Example 16: Verification of Activity Inhibition for a Different Type of Kinase, Using a Kinase Panel Assay

A total of 430 kinds of kinases were treated with 1 μM of LDD3959 through a kinase panel assay conducted by Eurofins to examine each activity.

Among them, for 4 kinases that specifically showed inhibition, the activity inhibition effect was examined with the highest concentration of 1 μM. The ATP concentration used in this experiment was 10 μM.

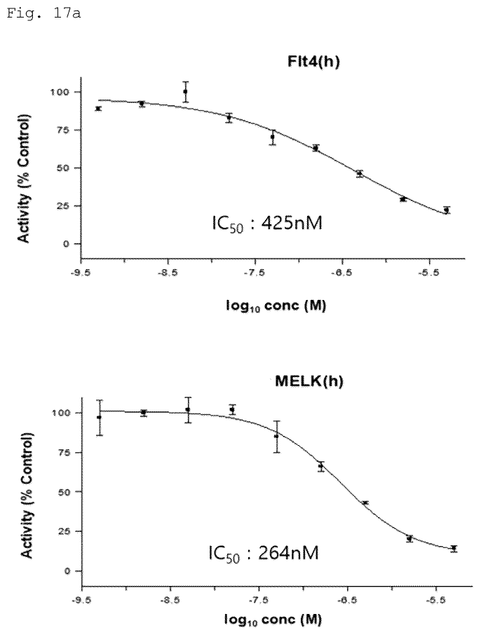

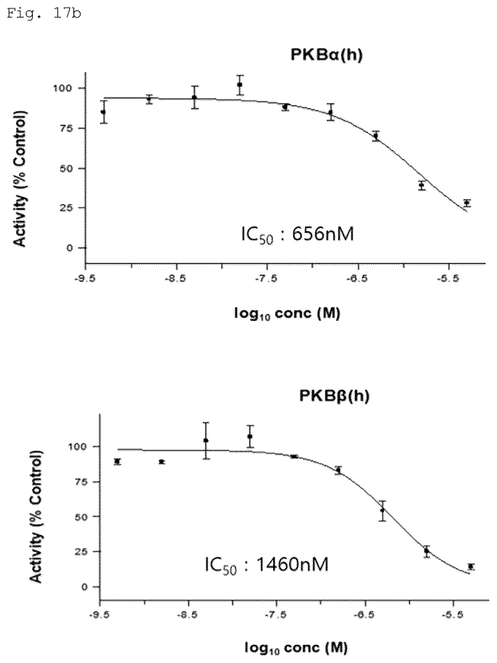

As shown in FIGS. 17a and 17b, the examination results verify that when the 4 kinases were treated with LDD3959, very good inhibitory activity was exhibited. Since it is known that all of the 4 kinases exhibit high activity in tumors, the results of this experiment verify that LDD3959 can be used as an anticancer agent.

### Experiment Example 17: Examination of Toxicity by Single Oral Administration

LDD3959 was orally administered once for each concentration in the range of 500 to 2000 mg/kg, and adverse events (body weight changes, feed intake changes, general appearance changes) were checked for 2 weeks. After 2 weeks of administration, the mice were sacrificed. Then, the mice underwent hematological examination and measurement of weight change of each organ thereof. In addition, the same experiment was performed on five ICR female mice and five ICR male mice to identify gender-dependent differences.

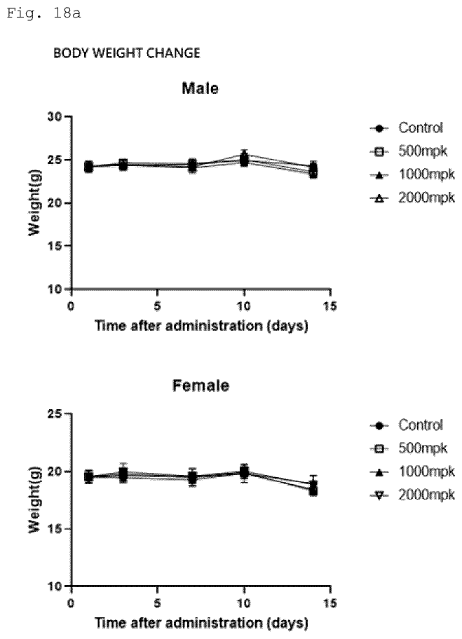

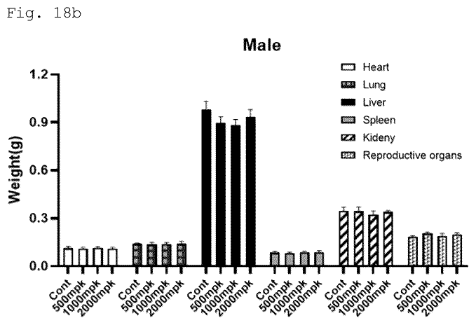

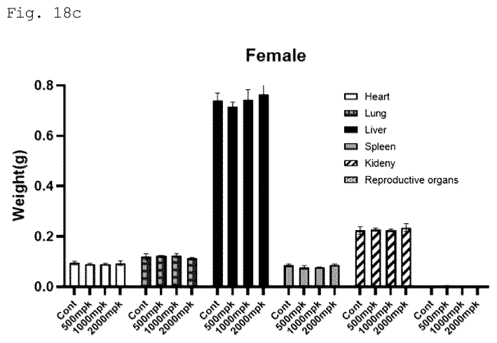

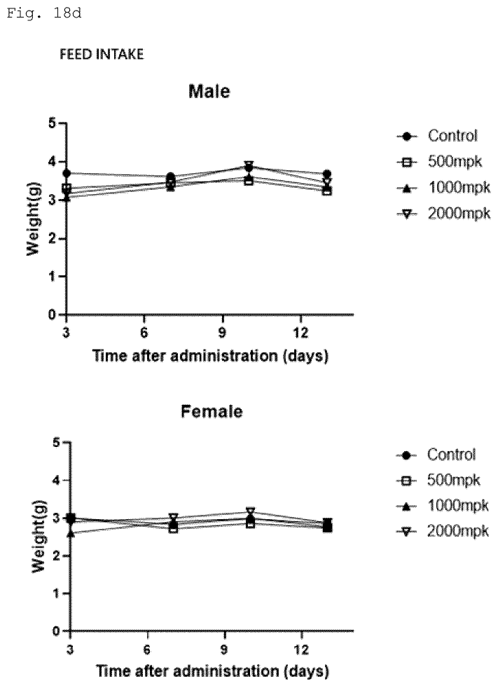

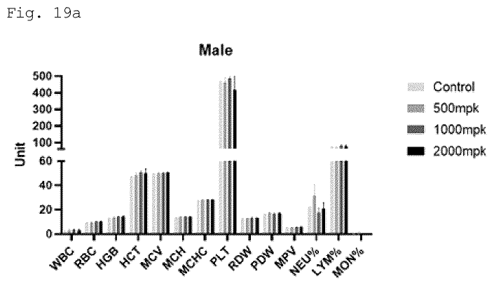

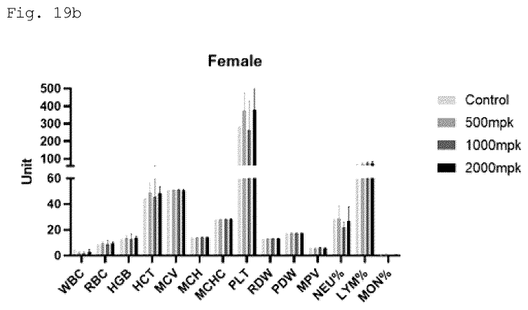

As a result, referring to FIGS. 18a to 18d and FIGS. 19a and 19b, no deaths of the subjects occurred by the single administration, and no specific adverse responses occurred in blood.

### Experiment Example 18: Examination of Toxicity by 3-Week DRF Oral Administration

LDD3959 was administered once a day for 3 weeks at each concentration (in the range of 500 to 2000 mg/kg), adverse events during the course of administration were checked, and the subjects were sacrificed the next day after the end of administration to perform weight measurement, hematological analysis, and biochemical analysis for each organ. In addition, the same experiment was performed on five ICR female mice and five ICR male mice to identify gender-dependent differences.

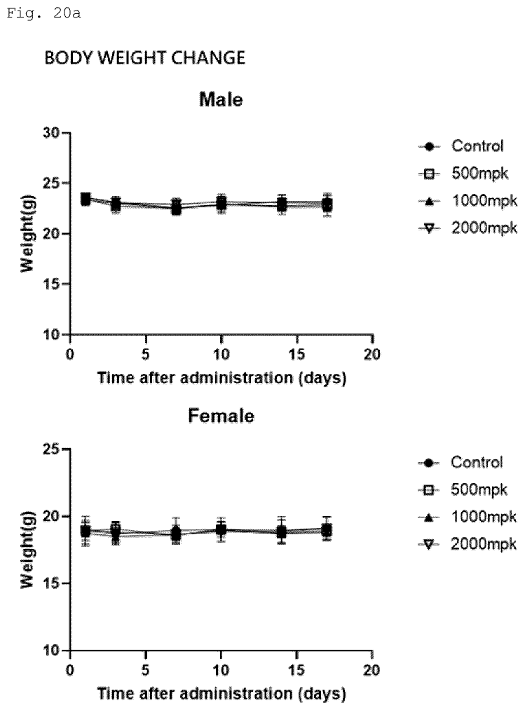

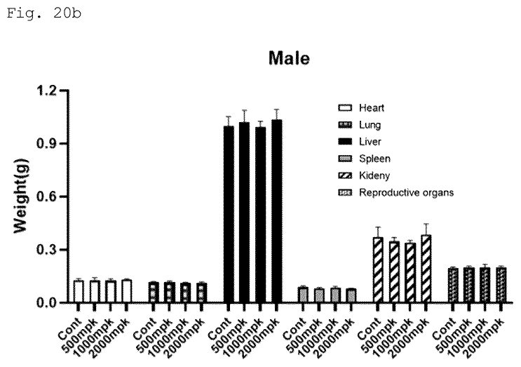

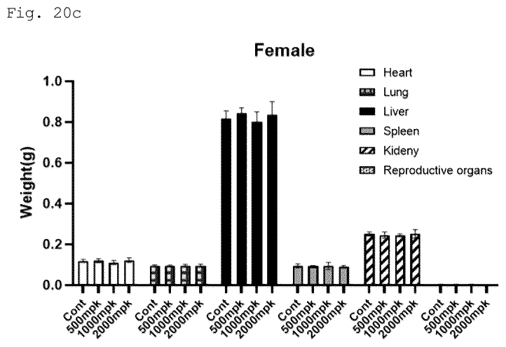

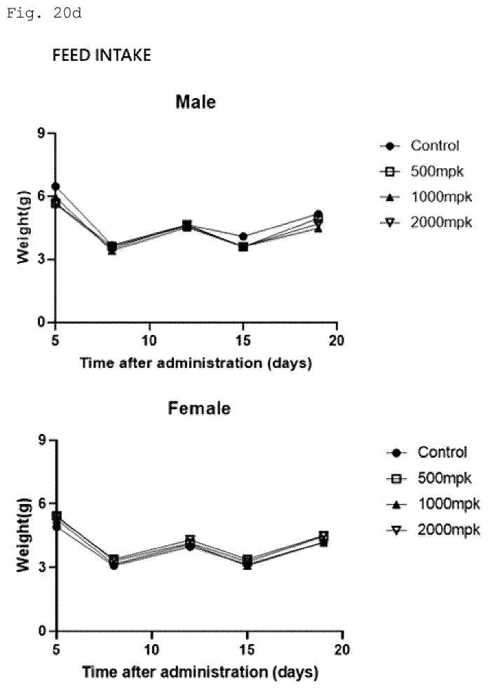

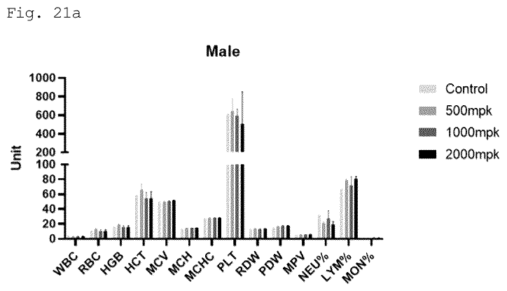

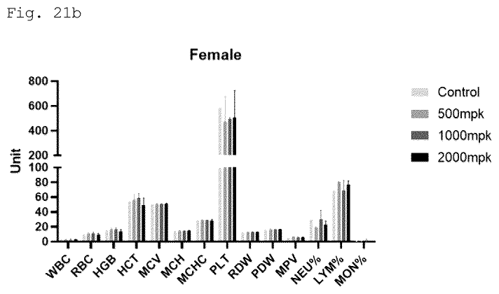

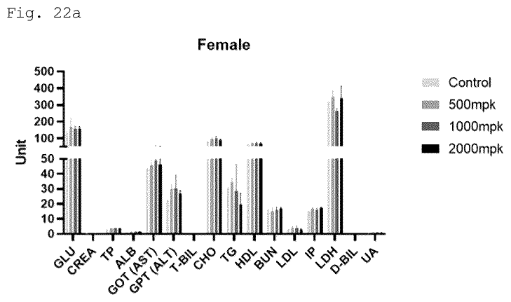

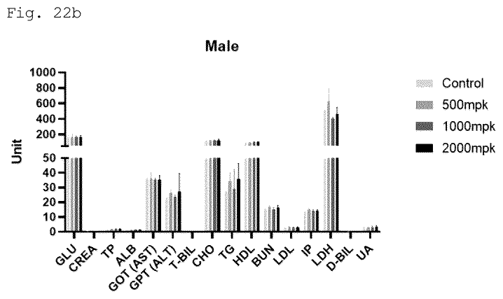

As a result, referring to FIGS. 20a to 20d and FIGS. 21a and 21b, no deaths of the subjects occurred by the 3-week daily administrations, and no specific adverse events occurred. In addition, the results of hematological and biochemical tests also showed no probable adverse events when the concentration was changed.

The ordinarily skilled people in the art will appreciate, from the detailed description, that the present invention can be implemented in other different forms without departing from the technical spirit or essential characteristics of the examples. Therefore, it should be understood that the examples described above are only for illustrative purposes and are not restrictive in all aspects. The scope of the present invention should be defined by the appended claims rather than the above detailed description and should be construed such that all changes or modifications derived from the meaning and scope of the claims and their equivalent concepts fall within the scope of the present invention.

1. A compound represented by Formula 1 or a pharmaceutically acceptable salt thereof: 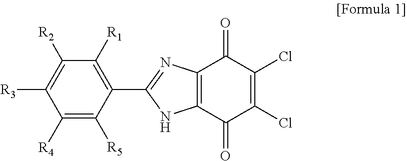
wherein in Formula 1, R₁ to R₅ are each independently hydrogen, halogen, cyano, nitro, amino, carboxyl, or carbamoyl, except that R₁ to R₅ are all hydrogen.

2. The compound or pharmaceutically acceptable salt of claim 1, wherein R₁ to R₅ are each independently hydrogen or halogen.

3. The compound or pharmaceutically acceptable salt of claim 1, wherein the compound is 2-(4-bromophenyl)-5,6-dichloro-1H-benzo[d]imidazole-4,7-dione or 2-(4-bromo-3-fluorophenyl)-5,6-dichloro-1H-benzo[d]imidazole-4,7-dione).

4. A pharmaceutical composition for preventing or treating transglutaminase 2-related diseases, the composition comprising a compound represented by Formula 1 or a pharmaceutically acceptable salt thereof: 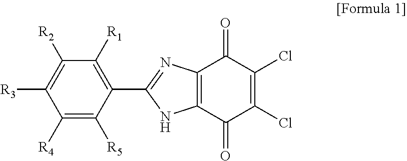
wherein in Formula 1, R₁ to R₅ are each independently hydrogen, halogen, cyano, nitro, amino, carboxyl, or carbamoyl.

5. The pharmaceutical composition of claim 4, comprising 5,6-dichloro-2-phenyl-1H-benzo[d]imidazole-4,7-dione, 2-(4-bromophenyl)-5,6-dichloro-1H-benzo[d]imidazole-4,7-dione, or 2-(4-bromo-3-fluorophenyl)-dichloro-1H-benzo[d]imidazole-4,7-dione, as an active ingredient.

6. The pharmaceutical composition of claim 4, wherein the transglutaminase 2-related diseases include fibroproliferative diseases, cancers, cardiovascular diseases (CVDs), celiac diseases, gastroenterological diseases, inflammatory diseases, neurological disorders, or idiopathic inflammatory myopathies, caused by abnormal expression or abnormal activity of transglutaminase 2.

7. The pharmaceutical composition of claim 4, wherein the pharmaceutical composition exhibits a prophylactic or therapeutic effect by inhibiting the activity of transglutaminase 2.

8. A transglutaminase 2 inhibitory composition comprising a compound represented by Formula 1 or a pharmaceutically acceptable salt thereof: 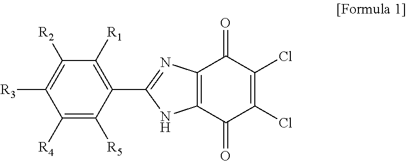
wherein in Formula 1, R₁ to R₅ are each independently hydrogen, halogen, cyano, nitro, amino, carboxyl, or carbamoyl.
